(19) United States
(12) Patent Application Publication
(10) Pub. No.: US 2015/0313993 A1
(43) Pub. Date: Nov. 5, 2015

(54) Ultrasonic Method and Device for Cosmetic Applications

(71) Applicant: Robert T. Bock, Brewster, NY (US)

(72) Inventor: Robert T. Bock, Brewster, NY (US)

(21) Appl. No.: 14/634,556

(22) Filed: Feb. 27, 2015

Related U.S. Application Data
(60) Provisional application No. 61/988,264, filed on May 4, 2014.
(60) Provisional application No. 62/119,921, filed on Feb. 24, 2015.

(51) Int. Cl. A61K 41/00; A61M 37/00

(52) U.S. Cl. CPC A61K 41/0047; A61M 37/0092; A61M 2037/0007; A61B 2017/00765

(57) ABSTRACT

A combination of low frequency high amplitude sonic frequency vibrations and high frequency low intensity ultrasonic pressure waves are applied to cosmetic compounds and to the skin to promote improved penetration of the cosmetic compounds into the epidermis. The cosmetic applicator device includes means for generating both sonic frequency vibrations and ultrasonic pressure waves adopted to deliver cosmetic compounds into the epidermis safely without significant temperature rise in the skin. Various removable applicator and skin cleaning attachments are also disclosed.

### FIELD OF THE INVENTION

This invention relates to sonic and/or ultrasonic devices and methods for cosmetic applications.

### BACKGROUND OF THE INVENTION

The stratum corneum, the outermost layer of the epidermis consists of dead cells (corneocytes). The purpose of this layer of dead skin is to form a barrier to protect underlying living tissue from infection, dehydration, and chemical attacks.

Unfortunately, the same low permeability barrier characteristic of the stratum corneum, which protects the body from infections, also resists the penetration of beneficial cosmetic and chemical compounds, such as moisturizers, alpha-hydroxyl acids, collagen, vitamins and vasodilators. In addition, oily and congested skin conditions are also reducing the penetration of beneficial skin treatment compounds.

The invention is concerned with methods and apparatus facilitating the use of sonic and ultrasonic energy coupled to the skin to temporarily increase the permeability of the skin and enhance the absorption of beneficial cosmetic and chemical compounds into the skin.

### DESCRIPTION OF PRIOR ART

Numerous attempts have been made in the past to enhance the penetrations of cosmetic compounds into the skin by chemical, electrical and ultrasonic means.

The application of chemicals to modify the skin structure to allow the penetration of cosmetics was found to be dangerous because while it provided access for cosmetics to penetrate, it left the body unprotected against harmful environments, interacting with corneocytes causing irritation, erythema (red skin) and contact dermatitis.

The application of electrical fields to create transient transport pathways by a method called electroporation, and the method to electrically charge molecules to increase their penetration into the skin called iontophoresis (U.S. Pat. No. 6,169,920), have both been proven costly and ineffective. Electrical abrasion devices for increasing the skin's permeability (U.S. Pat. No. 8,386,027) remove some layers of the stratum corneum causing intense irritation and discomfort.

The effort of prior art of ultrasonically induced drug delivery (sonophoresis) described in U.S. Pat. No. 6,322,532 is focused in driving drug molecules through the skin by high frequency and high intensity ultrasonic pressure waves. This procedure suffers from the disadvantage of tissue heating and the associated modification and sometimes destruction of healthy cells.

Notwithstanding the teaching of the prior art, the ability to deliver cosmetic compounds into the skin below the stratum corneum safely, effectively, inexpensively and easily has remained unsolved.

Responding to the above described unresolved needs, the object of this invention is to provide a skin care method and apparatus to safely increase the permeability of the stratum corneum and deliver cosmetic compounds deeply into the dermis.

### SUMMARY OF THE INVENTION

As noted in the description of prior art, the safety of the typical sonophoresis apparatus is compromised by the high intensity requirements of the process, resulting in excessive tissue heating and its associated consequences.

The objective of the invention is to improve the safety of typical sonophoresis apparatus to deliver cosmetic compounds into the dermis at reduced ultrasound intensity.

The method of the invention achieves this objective of utilizing lower intensity ultrasonic pressure waves by augmenting the ultrasonic pressure waves with non-tissue heating low frequency sonic vibrations applied to the skin in combination with the high frequency ultrasound. The low frequency sonic vibration component of this new method increases the permeability of the skin and allows a lower intensity non-tissue heating ultrasound component to drive the cosmetic compound through the stratum corneum into the dermis. Furthermore, since oils and various contaminants on the skin can reduce the penetration of cosmetic compounds, an optional pre treatment skin-cleansing step is part of the disclosed method.

A novel apparatus is provided having the capability to simultaneously generate tactile sonic frequency vibrations and ultrasonic pressure waves and apply them to the skin. To emphasize, the key characteristic of the invention is the synergistic application of tactile sonic frequency vibrations with the ultrasonic pressure waves, which allow the utilization of lower intensity non-tissue heating ultrasound to facilitate the penetration of and to drive cosmetic compounds safely into the dermis.

In the above discussion, the terms cosmetic compounds and vasodilators includes but not limited to skin care products such as anti wrinkle lotions, moisturizers, antioxidant vitamins, alpha-hydroxyl acids, liposomes, collagen, elastin, hair growth and hair remover compounds and others.

### BRIEF DESCRIPTION OF THE DRAWINGS

FIG. 1 shows a longitudinal cross section of the invention consisting of the device handle, the motion transducer neck, the applicator portion including an ultrasonic transducer, the driving motor, electronic controls and battery.

FIG. 2 shows the cross section of the neck of the device, which is configured to act as a motion transducer.

FIG. 3A shows the applicator head of the device in contact with the skin.

FIG. 3B illustrates the sonic frequency component of the device and its effects on the stratum corneum.

FIG. 4 illustrates the simultaneous application of the sonic frequency vibration and ultrasound pressure wave components of the device and their combined effects on the stratum corneum.

FIG. 5 shows a longitudinal cross section of an alternative configuration of the invention.

FIG. 6 shows a removable applicator head designed for convex areas of the anatomy.

FIG. 7 shows a removable applicator head designed for concave areas of the anatomy.

FIG. 8 shows a removable brush head for cleansing the skin.

### DESCRIPTION OF THE PREFERRED EMBODIMENTS

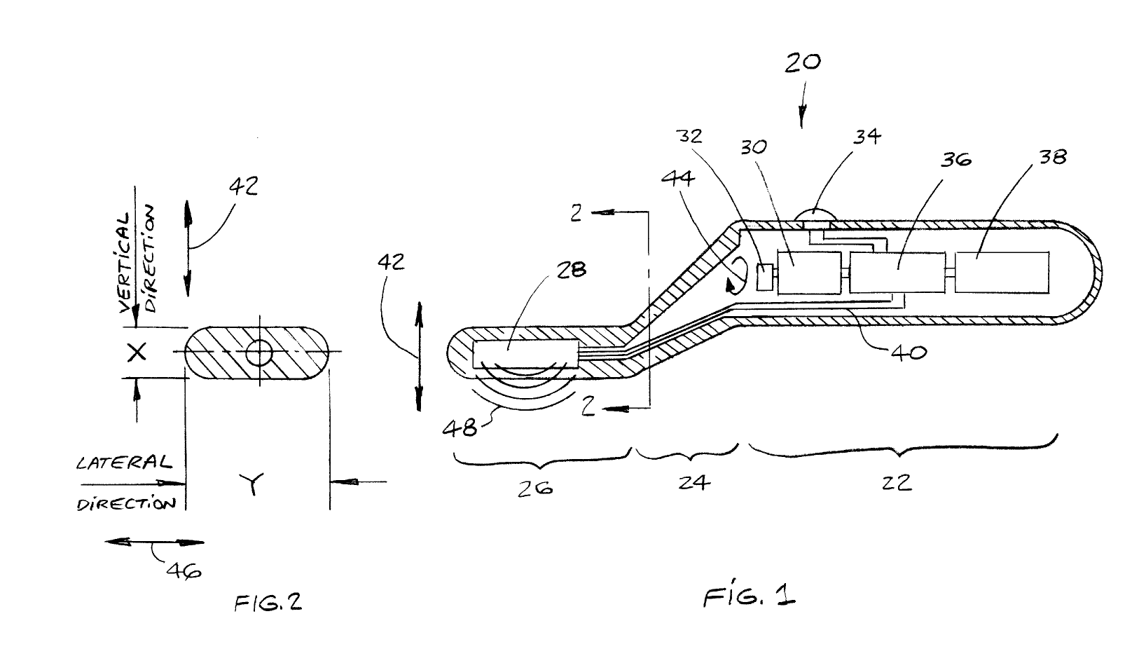

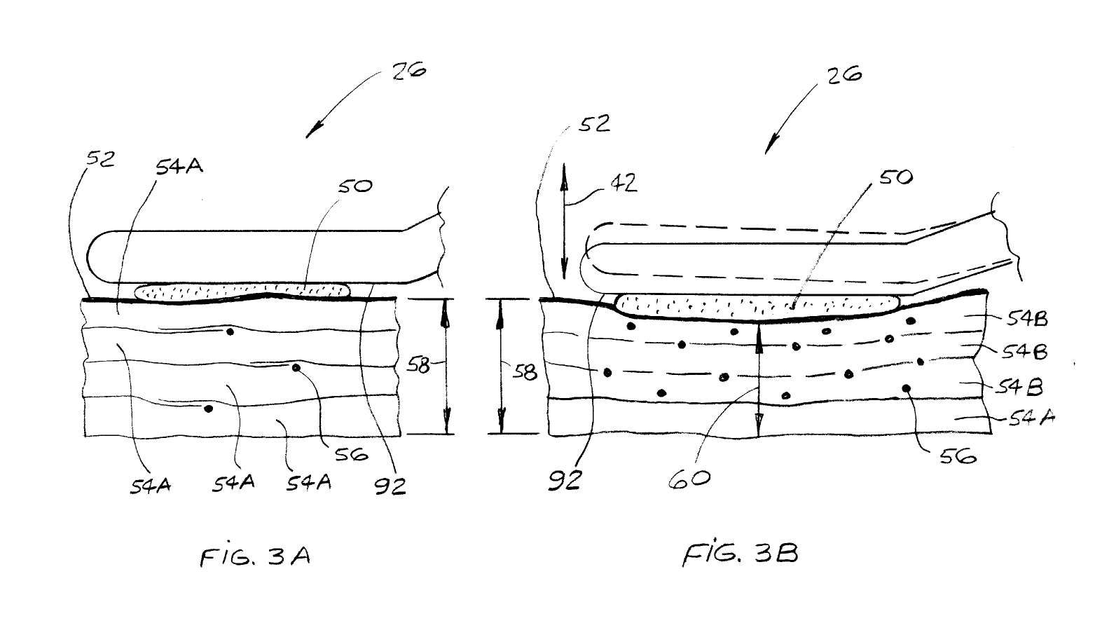

FIG. 1 and FIG. 2 show the invention of the ultrasonic cosmetic applicator 20 in a preferred configuration. The applicator 20 comprises a tubular shaped handle portion 22, a neck portion 24, and an applicator head portion 26 constructed of a rigid plastic material such as Acrylonitrile Butadiene Styrene (ABS), an ultrasound transducer 28, a driving motor 30, an eccentric weight 32 mounted on the output shaft of the driving motor 30, an electronic module 36, a battery pack 38, and interconnecting wiring 40.

The ultrasound transducer 28 is typically constructed of a piezo-electric ceramic material such as PZT-8 grade Lead Zirconate Titanate manufactured by Morgan Matroc, Inc., or similar products manufactured by numerous other entities. The construction of the ultrasound transducer 28 can be a single or a multiple element unit, as it is commonly practiced by people familiar in the art.

The ABS material utilized for the applicator 20 is due to the ABS excellent acoustic characteristics. However, numerous other rigid plastic materials could be substituted to achieve various cost and performance goals of the designers.

Control switch 34 energizes the driving motor 30, which rotates the eccentrically mounted weight 32 between 2,000 and 25,000 RPM, ideal speed being at 9,000 RPM, generating a 33 to 417 Hertz sonic frequency rotational vibration 44 of the handle 22 and neck 24 portions of the applicator 20, which is considered a relatively low sonic frequency vibration in the art, which defines sonic frequency vibration as being 10 to 20,000 Hertz. As shown in FIG. 2 the cross section of the neck 24 is designed to be relatively thin in the vertical direction X compared to the lateral direction Y thereby significantly increasing the vertical vibration 42 amplitude of the applicator head 26 while significantly decreasing lateral vibration 46 amplitude of the applicator head 26. In other words, the neck portion 24 of the applicator 20 is designed to be a motion transducer to convert the rotational vibration 44 of the handle 22 portion of the applicator 20 into a substantially vertical vibration 42 of the applicator head 26, converting the rotational energy of the motor 30 into vertically vibrating energy of the applicator head 26.

The battery pack 38 can be constructed as a single cell or multi cell battery pack, of various chemistries, such as Alkaline Manganese, Nickel-Cadmium, Ni—Mh, Lithium or other newer construction.

The major function of the electronic module 36 is to convert the low voltage DC power, typically 1.5 to 4.8 VDC, of the battery pack 38 into high voltage (4.8 to 60 Volt) typically sinusoidal wave ultrasonic frequency (typically 15 kHz to 20 MHz) DC power in a continuous wave or burst wave modality.

Simultaneously with energizing the driving motor 30, switch 34 also activates the electronic module 36. Through the interconnecting wiring 40 the electronic module 36 energizes the ultrasound transducer 28 which contracts and expands in tune with the high frequency DC power and converts this electronic power into ultrasonic pressure waves 48 at a typical intensity from 0.05 to 0.5 W/cm².

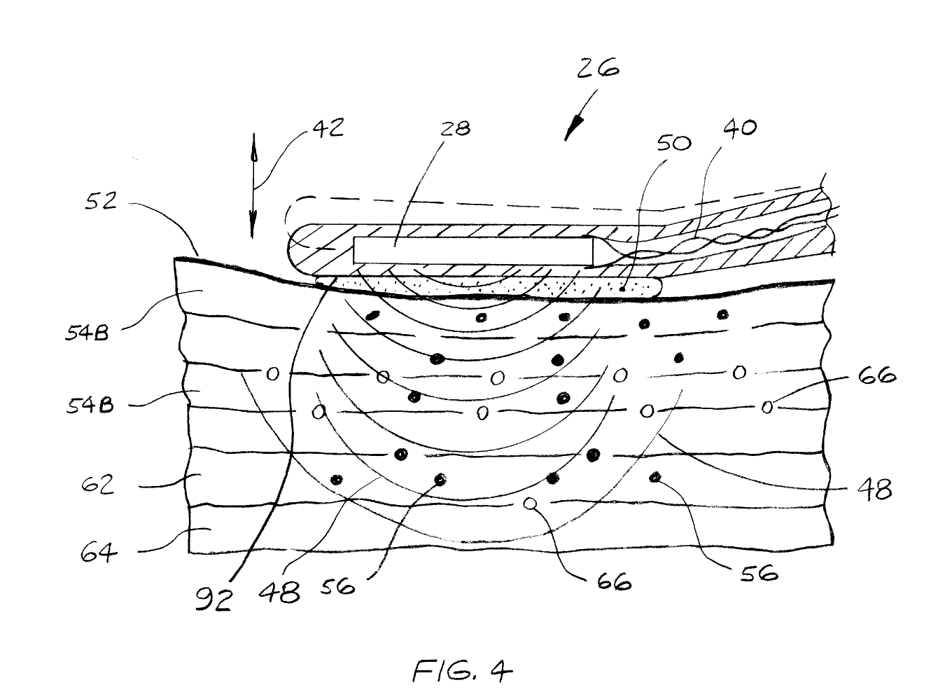

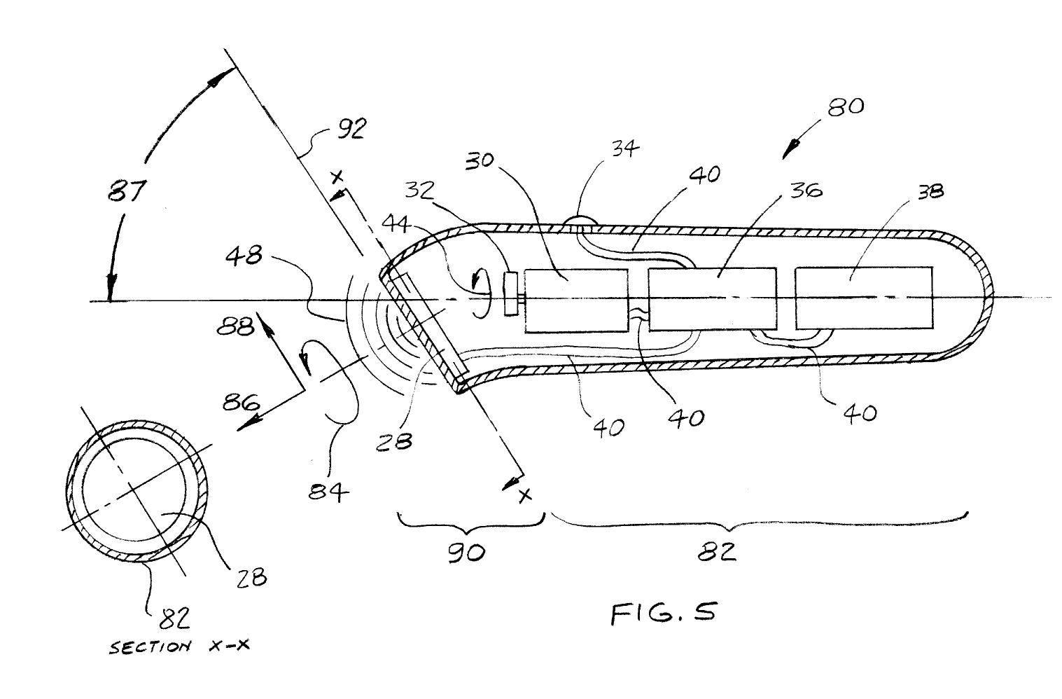

In FIG. 3A the applicator head 26 of the applicator 20 is shown in position on top of the outer surface of the stratum corneum 52, consisting of flat dead cells filled with keratin fibers surrounded by ordered lipid bilayers 54A shown in a relaxed position 58. The ordered structure of the stratum corneum 52 and the ordered lipid bilayers 54A are forming a normally almost impermeable skin structure. A thin layer of cosmetic compound 50 is shown to be disposed between the applicator contact surface 92 of the applicator head 26 and the stratum corneum 52. A typically very limited amount of small molecules 56 of the cosmetic compound 50 are shown to be penetrating slightly into the ordered lipid bilayers 54A without assistance from the applicator head 26.

FIG. 3B shows the applicator head 26 activated in the vertically vibrating 42 mode on top of the stratum corneum 52 and a thin layer of cosmetic compound 50 is shown to be disposed between the applicator contact surface 92 of the applicator head 26 and the stratum corneum 52. The vertical vibration 42 of the applicator head 26 (also depicted with solid and dashed lines to illustrate vibration) repeatedly compresses and relaxes the stratum corneum 52 and the ordered lipid bilayers 54A from the relaxed position 58 to the compressed position 60 in tune with the high amplitude low frequency vibration mode of the applicator head 26. Under the repeated and continuing influence of this high amplitude low sonic frequency vibration 42 and the resulting repeated compression and relaxation cycles of the stratum corneum 52 and the ordered lipid bilayers 54A, the ordered lipid bilayers 54A beginning to disorganize and develop larger passage ways for the molecules 56 of the cosmetic compound 50 to pass through. The disorganized lipid bilayers 54B are depicted with dashed lines.

FIG. 4 shows the applicator head 26 in contact with the stratum corneum 52 while having a thin layer of cosmetic compound 50 disposed between the applicator contact surface 92 of the applicator head 26 and the stratum corneum 52. The ultrasound transducer 28 is shown being energized by the electronic module 36 through the connective wiring 40 and radiating ultrasonic pressure waves 48 into the stratum corneum 52 and the disorganized lipid bilayers 54B. While the sonophoresis art has been demonstrated to work in the frequency range of 20 kHz to 20 MHz and in both of a continuous wave and a burst wave modality, it is important to select the right combination of frequency, driving voltage, and modality to match the size and characteristics of the piezo electric transducer selected for the system. Hard piezo materials such as the PZT8 formulation will output high ultrasonic power intensities with the associated heating of tissues when driven by high voltages. To avoid overheating the tissue, a 20% duty cycle (20% on 80% off) burst modality has been proven helpful in prior art.

Now, according to the invention, safety of the sonophoresis process can be further enhanced by the simultaneous application of a non tissue heating high amplitude low sonic frequency mechanical vibration 42 and the ultrasonic pressure waves 48 to the stratum corneum 52. Due to the presence of the high amplitude low sonic frequency vibration 42 applied to the stratum corneum 52, which establishes the initial pathways through the stratum corneum 52, the intensity of the ultrasonic pressure waves 48 can be reduced significantly, resulting in proportional reduction of tissue heating, while maintaining the effectiveness of the process.

The high frequency ultrasonic pressure waves 48, as shown in FIG. 4, penetrate the disorganized lipid bilayers 54B much deeper than the lower sonic frequency vibrations 42 do. These ultrasonic pressure waves 48 in a preferred frequency range of 20 kHz to 2 MHz and in a 20% duty cycle burst modality are developing mild cavitation deep within the lipid bilayers 54B resulting in microscopic air and/or vacuum pockets 66 which act to further break up the organized lipid bilayers 54A shown in FIG. 3A into disorganized lipid bilayers 54B, generating more and deeper passage ways for the cosmetic compound molecules 56 to penetrate through the stratum corneum 52, through the disorganized lipid bilayers 54B, through the bottom layer of the epidermis 62 and into the dermis 64.

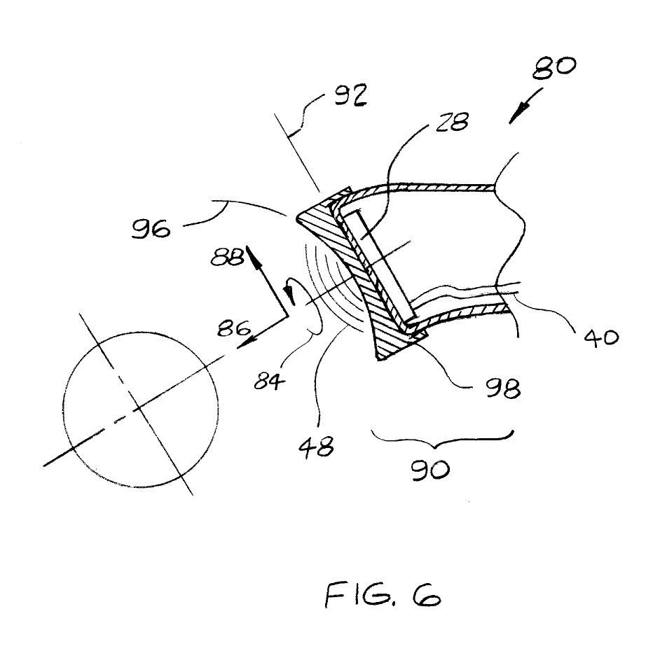

FIG. 5 shows a longitudinal cross section of an alternative configuration of the invention wherein the applicator 80 comprises a tubular shaped handle portion 82 terminating in an angular applicator head portion 90 constructed of a rigid plastic material such as Acrylonitrile Butadiene Styrene (ABS), an ultrasound transducer 28, a driving motor 30, an eccentric weight 32 mounted on the output shaft of the driving motor 30, an electronic module 36, a battery pack 38, and interconnecting wiring 40.

The ultrasound transducer 28 is typically constructed of a piezo-electric ceramic material such as PZT-8 grade Lead Zirconate Titanate manufactured by Morgan Matroc, Inc., or similar products manufactured by numerous other entities. The construction of the ultrasound transducer 28 can be a single or a multiple element unit, as it is commonly practiced by people familiar in the art.

The ABS material utilized for the applicator 80 is due to the ABS excellent acoustic characteristics. However, numerous other materials could be substituted to achieve various cost and performance goals of the designers. For example, the applicator contact surface 92 may be constructed of stainless steel or other metallic material.

Control switch 34 energizes the driving motor 30, which rotates the eccentrically mounted weight 32 between 2,000 and 25,000 RPM, ideal speed being at 9,000 RPM, generating a 33 to 417 Hertz sonic frequency rotational vibration 44 of the handle portion 82 of the applicator 80.

The angular positioning 87 of the applicator contact surface 92 of the applicator head portion 90 acts as a motion transducer converting the rotational vibration 44 of the handle portion 82 into an angular rotational vibration 84 of the applicator contact surface 92 of the applicator head portion 90. The angular rotational vibration 84 creates a two dimensional vibration motion of the applicator contact surface 92 in the directions of motion vector 86 and motion vector 88.

While FIG. 5 depicts an angularly fixed applicator head portion 90 construction, applicator 80 can also be constructed having a user adjustable angular applicator head portion 90 wherein the user can vary the angular positioning 87 of the applicator contact surface 92 to increase or decrease the vibratory motion in the directions of motion vector 86 and motion vector 88. A decreasing angle 87 will decrease the vibration amplitude of motion vector 88 and increase the vibration amplitude of motion vector 86.

The battery pack 38 can be constructed as a single cell or multi cell battery pack, of various chemistries, such as Alkaline Manganese, Nickel-Cadmium, Ni—Mh, Lithium or other newer construction.

The major function of the electronic module 36 is to convert the low voltage DC power, typically 1.5 to 4.8 VDC, of the battery pack 38 into high voltage (4.8 to 60 Volt) typically sinusoidal wave ultrasonic frequency (typically 15 kHz to 20 MHz) DC power in a continuous wave or burst wave modality.

Simultaneously with energizing the driving motor 30, switch 34 also activates the electronic module 36. Through the interconnecting wiring 40 the electronic module 36 energizes the ultrasound transducer 28 which contracts and expands in tune with the high frequency DC power and converts this electronic power into ultrasonic pressure waves 48 at a typical intensity from 0.05 to 0.5 W/cm².

The embodiment of the invention as applicator 80 depicted in FIG. 5 functions the same way as the embodiment of the invention as applicator 20 depicted in FIGS. 1, 2, 3A, 3B, and 4. More particularly, the sonic frequency vibration of the applicator contact surface 92 of the applicator head 90 in the direction of motion vector 86 described in FIG. 5 functions the same way as the sonic frequency vibration of the applicator contact surface 92 of applicator head 26 in the direction of motion vector 42 described in FIG. 3B and FIG. 4. The ultrasonic pressure waves 48 radiated from applicator 80 described in FIG. 5 function the same way as the ultrasonic pressure waves 48 radiated from applicator head 26 described in FIG. 4. The underlying science of the two embodiments are identical.

FIG. 6 shows a removable applicator head 98 designed to conduct the low frequency orbital vibration 84 and vibration motion vectors 86 and 88 and the ultrasound pressure waves 48 into the hard convex areas of the anatomy, such as the scalp, the elbows, and similar areas.

The applicator contact surface 92 of the applicator head 90 as described earlier in FIG. 5 is typically made of rigid or semi rigid material designed for soft flexible surfaces of the anatomy, such as the cheeks, where the anatomy conforms to the applicator contact surface 92 under slight pressure and transmission of the ultrasonic pressure waves 48 to the anatomy is easily achieved. However, when the flat rigid applicator contact surface 92 is applied to a hard convex area, such as the scalp, it results in a very small single point contact, which limits the transmission of the ultrasonic pressure waves to the anatomy.

To maximize transmission of the ultrasonic pressure waves 48 to the hard convex areas of the anatomy the removable applicator head 98 is made of a flexible ultrasound conductive material such as silicone rubber and features a concave contact surface 96 which easily conforms to the anatomy under slight pressure. To further assure excellent transmission of the ultrasound pressure waves 48 from the ultrasound transducer 28 to the removable applicator head 98 a slight coating of ultrasound conductive material such as water or gel can be applied between the applicator contact surface 92 and the removable applicator head 98.

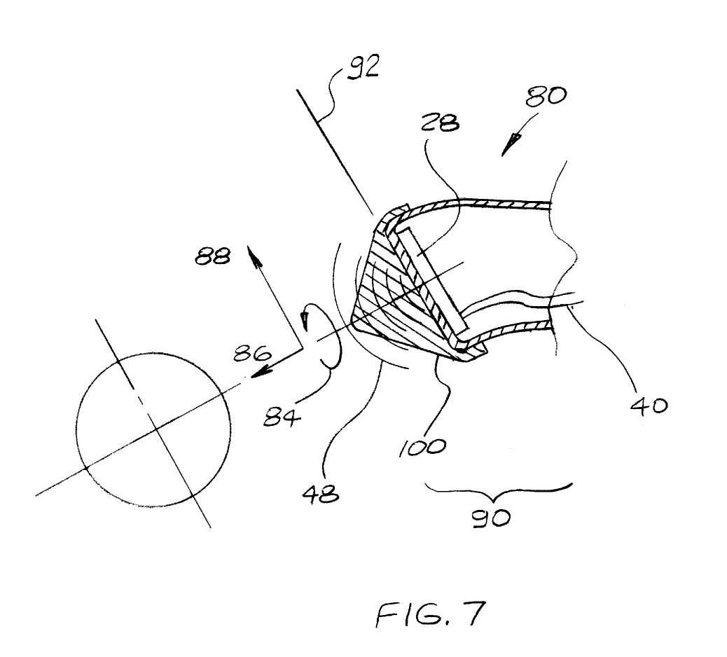

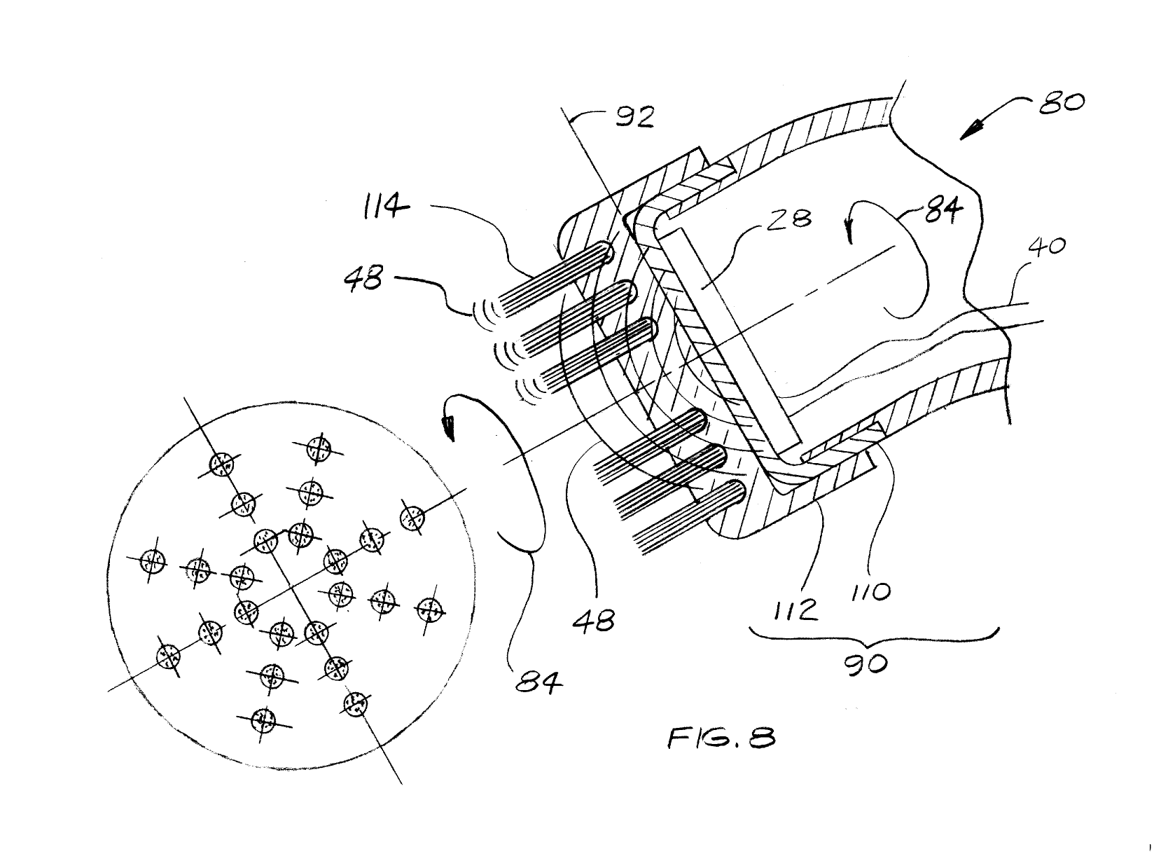

FIG. 7 shows a removable applicator head 100 designed for concave areas of the anatomy. Such small concave areas as between the eyes and the nose or between the cheeks and the nose are typically not accessible by the applicator contact surface 92 designed for larger soft surfaces of the anatomy. The removable applicator head 100 is constructed of rigid or semi rigid materials, such as ABS, or flexible silicone rubber conducting the low frequency orbital vibration 84 and vibration motion vectors 86 and 88 and the ultrasound pressure waves 48 into these small concave areas.

FIG. 8 shows a removable cleansing brush head 112 installed on the applicator head portion 90 of applicator 80. The brush head 112 is typically constructed of a semi rigid ABS plastic material housing multiple tufts of bristles 114. As described in detail in FIG. 5 the applicator motor 30 vibrates the applicator head portion 90 in an orbital vibration 84 pattern. This orbital vibration 84 is transferred to the brush head 112 and the plurality of bristle tufts 114. When energized through the interconnecting wiring 40 the ultrasound transducer 28 generates and emits ultrasound pressure waves 48 which are conducted by the applicator contact surface 92 to the brush head 112 and the bristle tufts 114 and radiated from the bristle tufts 114 to the skin of the user. Applying slight pressure of the orbitally vibrating 84 bristle tufts 114 against the skin the user effectively cleansing the skin by the synergistic scrubbing action of the bristle tufts 114 and the ultrasound pressure waves 48 radiated by the bristle tufts 114.

FIG. 8 also shows an optional construction of the applicator head portion 90 incorporating a stainless still cup 110.

While the preceding description contains much specificity, these should not be construed as limitations on the scope of the invention, but rather as an exemplification of preferred and additional embodiments thereof. Skilled artisans will readily be able to change dimensions, shapes, and construction materials of the various components described in the embodiments and adopt the invention to various types of sonic and ultrasonic energy applications. For example, additional removable and interchangeable applicators for enhanced cleansing of the skin such as sponges, cotton pads, lotion dispensers enhanced by the sonic and ultrasonic frequency motion of the applicator head are possible. Accordingly, the scope of the invention should be determined not by the embodiments illustrated, but by the appended claims and their legal equivalents.

What is claimed:

1. An improved method of facilitating the penetration of cosmetic or chemical compounds into a person's skin comprising, applying said compound to the skin, then applying sonic frequency vibrations to said compound and said skin to disorganize the top layer of the stratum corneum and the lipid bilayers and to safely increase the permeability of said skin, concurrently applying ultrasonic pressure waves to said compound and said skin of sufficiently high intensity to cause mild cavitation in said skin thereby opening up deeper passageways through said stratum corneum by further disordering said lipid bilayers and further increasing the permeability of said skin to allow the deeper penetration of the molecules of said compound applied to said skin.

2. The method as defined in claim 1 wherein the sonic frequency vibrations applied in the range of about 33 to about 250 Hz, and the ultrasonic pressure waves are applied in the range of about 15 kHz to about 20 MHz in a continuous wave modality.

3. The method as defined in claim 1 wherein the sonic frequency vibrations applied in the range of about 33 to about 250 Hz, and the ultrasonic pressure waves are applied in the range of about 15 kHz to about 20 MHz in a pulsed wave modality.

4. The method of claim 2 or 3, wherein said skin is cleaned by a device powered by low sonic frequency vibrations in combination with ultrasonic pressure waves prior to the application of said compound to the skin.

5. A device to improve penetration of cosmetic or chemical compounds into the skin comprising, a handle end and an applicator end, means to generate sonic frequency vibrations of said applicator end operative to increase permeability of said skin, an ultrasound transducer located in the applicator end, means to generate and connect ultrasonic frequency electric signals to said ultrasound transducer, said ultrasound transducer generating ultrasonic pressure waves when energized by said ultrasonic frequency electric signals operative to transmit said ultrasonic pressure waves from said applicator end into said skin operative to increase permeability of said skin and to increase penetration of said compound into said skin.

6. The device of claim 5 further comprising a battery supplying power to said means to generate ultrasonic frequency electric signals and said means to generate sonic frequency vibrations of said applicator end.

7. A device as defined in claim 5 or 6 wherein the sonic frequency vibrations of the applicator end are in the range of about 33 to about 250 Hz, and the ultrasonic pressure waves generated by the piezoelectric transducer are in the range of about 15 kHz to about 20 MHz in a continuous wave modality.

8. A device as defined in claim 5 or 6 wherein the sonic frequency vibrations of the applicator end are in the range of about 33 to about 250 Hz, and the ultrasonic pressure waves generated by the piezoelectric transducer are in the range of about 15 kHz to about 20 MHz in a pulsed wave modality.

9. A device as defined in claim 7 or 8 wherein the sonic frequency vibrations of the applicator end are converted by motion transducer means into a relatively large vibration amplitude component perpendicular to the skin versus a relatively small vibration amplitude component of said sonic frequency vibrations parallel to the skin.

10. The device of claim 9 wherein the vibration amplitude conversion of the motion transducer means is variable by the user.

11. The device of claim 5 wherein the applicator contact surface is flat.

12. The device of claim 5 further comprising a removable applicator accessory having a concave contact surface.

13. The device of claim 5 further comprising a removable applicator accessory having a convex contact surface.

14. The device of claim 5 further comprising a removable brush accessory having at least one tuft of bristles utilizing said sonic and said ultrasonic frequency vibrations operative to cleanse said skin to further enhance penetration of said compounds into said skin.
